(19) United States
(12) Patent Application Publication
(10) Pub. No.: US 2026/0117436 A1
(43) Pub. Date: Apr. 30, 2026

(54) MELTBLOWN MATERIAL FORMED FROM BICOMPONENT POLYOLEFIN AND POLYESTER FIBERS

(71) Applicant: O&M Halyard, Inc., Mechanicsville, VA (US)

(72) Inventors: Ryan Solan, Atlanta, GA (US); Nimia Zoe Maya, Dunwoody, GA (US); Prasad Potnis, Johns Creek, GA (US)

(21) Appl. No.: 19/234,465

(22) Filed: Jun. 11, 2025

Related U.S. Application Data
(60) Provisional application No. 63/714,404, filed on Oct. 31, 2024.

(51) Int. Cl. D04H 1/4382; A61L 2/08; B01D 39/16; B32B 5/02; B32B 5/08; B32B 5/26; C08L 67/03; D01F 1/10; D01F 8/06; D01F 8/14; D04H 1/4291; D04H 1/435; D04H 3/16

(52) U.S. Cl. CPC D04H 1/43828; A61L 2/081; B01D 39/1623; B32B 5/022; B32B 5/08; B32B 5/269; C08L 67/03; D01F 1/10; D01F 8/06; D01F 8/14; D04H 1/4291; D04H 1/435; D04H 3/16; A61L 2103/50; B01D 2239/0233; B01D 2239/0435; B01D 2239/0618; B01D 2239/0622; B01D 2239/0627; B01D 2239/065; B32B 2250/20; B32B 2262/0253; B32B 2262/0284; B32B 2262/124; B32B 2535/00; D10B 2321/022; D10B 2501/04

(57) ABSTRACT

A meltblown material formed from bicomponent fibers that can withstand the effects of a gamma irradiation sterilization process without negatively impacting bacterial filtration, particulate filtration, and/or breathability of personal protective equipment products formed from the meltblown material (e.g., facemasks, surgical gowns, surgical caps, etc.) is provided. The bicomponent fibers include a polyolefin-based sheath and a polyester-based core. Further, both the sheath and core include specific antioxidants and charge enhancer packages that allow for the effective electret treatment of the resulting meltblown material prior to sterilization, where the filtration efficiency and pressure differential of the material are still adequate post-sterilization.

### CROSS-REFERENCE TO RELATED APPLICATIONS

The present application claims the benefit of U.S. Provisional Application No. 63/714,404, filed Oct. 31, 2024, the contents of which are incorporated herein by reference.

### TECHNICAL FIELD

The subject matter disclosed here in is generally directed to a meltblown material that can be subjected to gamma irradiation during a sterilization process without negatively impacting the properties of the meltblown material.

### BACKGROUND

Existing polyolefin-based meltblown products (e.g., polypropylene (PP) meltblown products) that are commercially available today experience high levels of degradation after exposure to gamma irradiation during sterilization processes, such is the sterilization of facemask, surgical gowns, and other personal protective equipment (PPE) formed from nonwoven materials. This often results in significant losses in particle filtration efficiency (PFE) and bacterial filtration efficiency (BFE), as well as undesirable increases in pressure differential of the materials, which corresponds with reduced breathability of the materials. In addition, such degradation can lead to an unpleasant odor. While electret treatment of meltblown products during the manufacturing process is an option to improve filtration efficiency stability over the life of the product, after gamma sterilization, there can be losses in static charge associated with the electret treatment that, in turn, lead to losses in filtration efficiency.

Meanwhile, polyester-based materials such as polyethylene terephthalate (PET) and polybutylene terephthalate (PBT), are able to more effectively withstand the degradation effects of gamma irradiation, but there are known challenges for electret treatment stability of polyester meltblown materials, as such materials have a higher tendency to lose static charge over time, leading to concerns with filtration efficiency for polyester-based products.

As such, a need currently exists for an improved nonwoven material, such as a meltblown material, that overcomes the various problems associated with polypropylene and polyester meltblown materials.

### SUMMARY

Aspects and advantages of embodiments of the present disclosure will be set forth in part in the following description, or can be learned from the description, or can be learned through practice of the embodiments.

In one aspect, an article that includes a nonwoven material is provided. The nonwoven material includes a plurality of bicomponent fibers each having a core and a sheath. Further, the core includes from about 80 wt. % to about 98 wt. % of an olefin homopolymer, from about 3 wt. % to about 7 wt. % of a first charge enhancer package, and from about 2 wt. % to about 6 wt. % of a first antioxidant package, based on the total weight of the core. In addition, the sheath includes from about 80 wt. % to about 98 wt. % of a thermoplastic polyester, from about 2 wt. % to about 6 wt. % a second charge enhancer package, and from about 3 wt. % to about 9 wt. % of a second antioxidant package based on a total weight of the sheath. Moreover, the nonwoven material exhibits a reduction in particle filtration efficiency after sterilization by gamma irradiation at a level of about 20 kilograys to about 30 kilograys of less than about 5%.

In another aspect, the olefin homopolymer can include polypropylene.

In yet another aspect, the olefin homopolymer can have a melt flow rate ranging from about 400 grams/10 minutes to about 1700 grams/10 minutes as determined at 2.16 kg and 230° C.

In an additional aspect, the olefin homopolymer can have a density ranging from about 0.86 grams/cm³ to about 0.94 g/cm³.

In a further aspect, the thermoplastic polyester can include polyethylene terephthalate.

In another aspect, the thermoplastic polyester can have an intrinsic viscosity ranging from about 0.50 dL/g to about 0.65 dL/g.

In one aspect, the first charge enhancer package can include a fatty acid metal salt and a fatty acid amide. In addition, the fatty acid metal salt can include a fatty acid portion and a metal portion. The fatty acid portion can include lauric acid, palmitic acid, stearic acid, oleic acid, or a combination thereof, and the metal portion can include magnesium, zinc, aluminum, or a combination thereof. Moreover, a ratio of the fatty acid metal salt to the fatty acid amide can range from about 3:1 to about 5:1.

In another aspect, the first antioxidant package can include a partially-hindered phenolic antioxidant, a hindered amine antioxidant, a phosphite-based antioxidant, or a combination thereof.

In still another aspect, the second charge enhancer package can include a fatty acid metal salt, barium titanium dioxide, or a combination thereof.

In yet another aspect, the second antioxidant package can include a phenolic antioxidant, a hindered amine antioxidant, a phenyl phosphite antioxidant, a hydroxyphenyl-triazine antioxidant, or a combination thereof.

In a further aspect, the nonwoven material can exhibit a particle filtration efficiency after sterilization by gamma irradiation at a level of about 20 kilograys to about 30 kilograys of at least about 95%.

In one more aspect, the nonwoven material can exhibit a bacterial filtration efficiency after sterilization by gamma irradiation at a level of about 20 kilograys to about 30 kilograys of at least about 94%.

In another aspect, the nonwoven material can exhibit a differential pressure (dP) after sterilization by gamma irradiation at a level of about 20 kilograys to about 30 kilograys of less than about 5 mm water/cm².

In still another aspect, the nonwoven material can include a meltblown material. In an additional aspect, the nonwoven material can include a laminate, and the laminate can include the meltblown material. Additionally, the laminate can further include a spunbond material.

In one more aspect, the nonwoven material can be electret-treated prior to sterilization.

In another aspect, the article can include a face mask, a surgical cap, or a surgical gown.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

A full and enabling disclosure of the present disclosure to one skilled in the art, including the best mode thereof, is set forth more particularly in the remainder of the specification, including reference to the accompanying figures, in which:

FIG. 1 is a cross-sectional view of a fiber contemplated by the present disclosure;

FIG. 2 is a top view of a nonwoven material formed from a plurality of fibers contemplated by the present disclosure;

FIG. 3 is a perspective view of a face mask having a body portion that can be formed from a material contemplated by the present disclosure;

FIG. 4 is a perspective view of a face mask with a body portion where the face mask is attached to the head of a user; and

FIG. 5 is a front view of a surgical gown that can be formed from a material contemplated by the present disclosure.

Repeat use of reference characters in the present specification and drawings is intended to represent the same or analogous features or elements of the present disclosure.

### DETAILED DESCRIPTION OF REPRESENTATIVE EMBODIMENTS

It is to be understood by one of ordinary skill in the art that the present discussion is a description of exemplary embodiments only, and is not intended as limiting the broader aspects of the present disclosure. Any of the features, components, or details of any of the arrangements or embodiments disclosed in this application are interchangeably combinable with any other features, components, or details of any of the arrangements or embodiments disclosed herein to form new arrangements and embodiments.

As used herein, the term “melt flow rate” (MFR) is measured according to ASTM D1238-13, entitled STANDARD TEST METHOD FOR MELT FLOW RATES OF THERMOPLASTICS BY EXTRUSION PLASTOMER and published in 2013 (“ASTM D1238”) (incorporated herein by reference). MFR is expressed (SI units) in g/10 min, and is the mass of polymer, in grams, flowing in ten minutes through a capillary of a specific diameter (typically about 2 mm) and length by a pressure applied via a range of standard weights (e.g., 2.16 kg) at a specified temperature (e.g., 230° C. for polypropylene). A polymer with a higher MFR typically has a lower viscosity and lower strength, while a polymer with a lower MFR typically has a higher viscosity and a higher strength.

As used herein, the term “meltblown web” generally refers to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, educative drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Petersen, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 microns, and are often between about 5 to about 20 microns.

Generally speaking, the present disclosure is directed to a meltblown material formed from bicomponent fibers that can withstand the effects of a gamma irradiation sterilization process without negatively impacting bacterial filtration, particulate filtration, and/or breathability of personal protective equipment products formed from the meltblown material (e.g., facemasks, surgical gowns, surgical caps, etc.). The bicomponent fibers include a polyolefin-based sheath and a polyester-based core. Further, both the sheath and core include specific antioxidants and charge enhancer packages that allow for the effective electret treatment of the resulting meltblown material prior to sterilization, where the filtration efficiency and pressure differential of the material are still adequate post-sterilization.

Without intending to be limited by any particular theory, the present inventors have found that by using a polyolefin-based fiber for the sheath and polyester-based fiber for forming the core of the bicomponent fiber, the polyester-based core can be stabilized by the polyolefin-based sheath, which is more compatible and stable with existing electret treating processes. In addition, the polyester-based core can help to limit degradation and reduce the resulting odor during gamma sterilization since it is better able to withstand such conditions compared to polyolefin-based materials. The polyester-based core can also provide structural integrity and more constant mechanical filtration post-sterilization. By using a sheath of polyolefin-based fibers to surround the polyester-based core, the resulting meltblown material can effectively be electret treated during the manufacturing process. Further, the present inventors have found that the specific types of polymers, charge enhancers, and antioxidants contained in both the core and sheath of the bicomponent fibers contemplated by the present disclosure, as well as their weight percentages and resulting ratios, can circumvent the issues associated with the use of polyester-based fibers for electret treatment and can improve the ability of the polyester-based fibers to hold the Corona charge, resulting in better filtration efficiencies and decreased pressure differential post-sterilization.

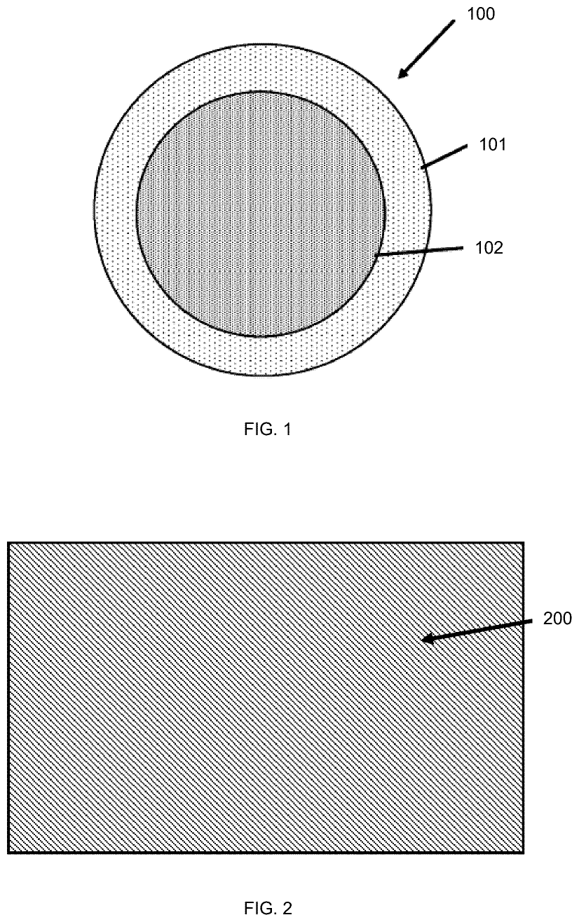

Referring now to FIGS. 1-5, the particular components of the present disclosure will be discussed in more detail. Specifically, FIG. 1 illustrates the fibers 100 from which the nonwoven material 200 (e.g., meltblown material) (see FIG. 2) for the PPE products of the present disclosure can be formed. As shown, the fibers 100 from which the nonwoven material 200 is formed can be multicomponent fibers, e.g., bicomponent fibers, and can have a sheath-core arrangement formed by a sheath 101 and a core 102.

Regardless of the specific polymer or polymers and additives used to form the multicomponent 100 fibers of the nonwoven material 200 described herein, the nonwoven material 200 can have a basis weight ranging from about 10 gsm to about 50 gsm, such as from about 15 gsm to about 45 gsm, such as from about 20 gsm to about 40 gsm.

In addition, before being subjected to sterilization, the nonwoven material can have a particle filtration efficiency (PFE) of at least about 99%, such as at least about 99.25%, such as at least about 99.5% for a single layer of nonwoven material, as measured by Latex Particle Challenge testing according to ASTM F2299. Further, after being subjected to sterilization via gamma irradiation (about 20 kilograys to about 30 kilograys for a time frame between about 8 hours and about 11 hours, such as about 9 hours to about 10 hours), the nonwoven material can have a particle filtration efficiency (PFE) of at least about 95%, such as at least about 95.5%, such as at least about 96% for a single layer of nonwoven material as measured by Latex Particle Challenge testing according to ASTM F2299. Compared to pre-sterilization, the reduction in PFE post-sterilization can be less than about 5%, such as less than about 4.75%, as such less than about 4.5%. In some embodiments, the reduction in PFE post-sterilization can be less than about 4.25%, such as less than about 4%. Moreover, it is to be understood that two or more layers of the nonwoven material can be bonded together to create a multi-layered nonwoven material, which can exhibit even further improved PFE compared to a single-layered nonwoven material.

Additionally, before being subjected to sterilization, the nonwoven material can have a bacterial filtration efficiency (BFE) of at least about 98%, such as at least about 98.5%, such as at least about 99%, as measured according to ASTM F2101. Further, after being subjected to sterilization via gamma irradiation (about 20 kilograys to about 30 kilograys for a time frame between about 8 hours and about 11 hours, such as about 9 hours to about 10 hours), the nonwoven material can have a bacterial filtration efficiency (BFE) of at least about 94%, such as at least about 94.5%, such as at least about 95%, such as at least about 95.2%, as measured according to ASTM F2101. Compared to pre-sterilization, the reduction in BFE post-sterilization can be less than about 5%, such as less than about 4.75%, such as less than about 4.5%. In some embodiments, the reduction in BFE post-sterilization can be less than about 3.25%, such as less than about 3%, such as less than about 2.75%. Moreover, it is to be understood that two or more layers of the nonwoven material can be bonded together to create a multi-layered nonwoven material, which can exhibit even further improved BFE compared to a single-layered nonwoven material.

Moreover, before being subjected to sterilization, the nonwoven material can exhibit a differential pressure (dP) of less than about 5 mm water/cm², such as less than about 4.75 mm water/cm², such as less than about 4.5 mm water/cm² as measured according to EN 14683:2019, Annex C to ensure the respiratory comfort and breathability of the nonwoven material product, where a lower dP indicates increased breathability. Desirably, the differential pressure is less than or equal to 5 mm water/cm². Further, after being subjected to sterilization via gamma irradiation (about 20 kilograys to about 30 kilograys for a time frame between about 8 hours and about 11 hours, such as about 9 hours to about 10 hours), the nonwoven material can also exhibit a differential pressure (dP) of less than about 5 mm water/cm², such as less than about 4.75 mm water/cm², such as less than about 4.5 mm water/cm² as measured according to EN 14683:2019, Annex C. Compared to pre-sterilization, the dP can decrease post-sterilization, indicating improvement in breathability, where the decrease in dP post sterilization can be greater than about 2%, such as greater than about 2.5%, such as greater than about 3%. In some embodiments, the decrease in dP post-sterilization can be greater than about 8%, such as greater than about 9%, such as greater than about 10%, such as greater than about 15%.

The various components of the sheath and core components of the fibers will now be discussed in more detail, followed by a discussion of the resulting properties of the nonwoven material formed from such fibers.

### Sheath

In some embodiments, the fibers 100 from which the nonwoven material is formed can have a sheath-core arrangement where the sheath 101 can include from about 80 wt. % to about 98 wt. %, such as from about 84 wt. % to about 96 wt. %, such as from about 88 wt. % to about 92 wt. % of an olefin homopolymer (e.g., polypropylene) based on the total weight of the sheath component of the multicomponent fiber. Particular polypropylene homopolymers that are contemplated by the present disclosure include ACHIEVE™ Advanced pp 6936G2 from ExxonMobil, TotalEnergies Polypropylene 3962 from Bamberger Amco Polymers, and Metocene MF650 W from LyondellBasell.

Any of a variety of known techniques may generally be employed to form the polyolefins. For instance, olefin polymers may be formed using a free radical or a coordination catalyst (e.g., Ziegler-Natta or metallocene). Metallocene-catalyzed polyolefins are described, for instance, in U.S. Pat. No. 5,571,619 to McAlpin et at; U.S. Pat. No. 5,322,728 to Davey, et al.; U.S. Pat. No. 5,472,775 to Obijeski et al.; U.S. Pat. No. 5,272,236 to Lai et al.; and U.S. Pat. No. 6,090,325 to Wheat, et al., which are incorporated herein in their entirety by reference thereto for all purposes. Examples of metallocene catalysts include bis(n-butylcyclopentadienyl) titanium dichloride, bis(n-butylcyclopentadienyl) zirconium dichloride, bis(cyclopentadienyl) scandium chloride, bis(indenyl) zirconium dichloride, bis(methylcyclopentadienyl) titanium dichloride, bis(methylcyclopentadienyl) zirconium dichloride, cobaltocene, cyclopentadienyltitanium trichloride, ferrocene, hafnocene dichloride, isopropyl(cyclopentadienyl,-1-flourenyl) zirconium dichloride, molybdocene dichloride, nickelocene, niobocene dichloride, ruthenocene, titanocene dichloride, zirconocene chloride hydride, zirconocene dichloride, and so forth. Polymers made using metallocene catalysts typically have a narrow molecular weight range. For instance, metallocene-catalyzed polymers may have polydispersity numbers (Mw/Mn) of below 4, controlled short chain branching distribution, and controlled isotacticity.

The melt flow rate (MFR) of the polypropylene homopolymer can range from about 400 grams/10 minutes to about 1700 grams per 10 minutes, such as from about 600 grams/10 minutes to about 1650 grams per 10 minutes about 0.1 grams per 10 minutes to about 100 grams per 10 minutes, such as from about 800 grams per 10 minutes to about 1600 grams per 10 minutes, as determined at 230° C. The melt flow rate is the weight of the polymer (in grams) that may be forced through an extrusion rheometer orifice (0.0825-inch diameter) when subjected to a force of 2.16 kg grams in 10 minutes at 230° C., and may be determined in accordance with ISO 1133. Moreover, the present inventors have found that because polypropylene and polyethylene terephthalate (PET) (discussed in more detail below) have such differing properties, using a lower melt flow rate polypropylene within the ranges described above while also controlling the intrinsic viscosity of the PET as outlined in the ranges below, allows for improved compatibility between the two polymers in order to optimize flow and mixing characteristics of the materials that were extruded through a die together to form the biocomponent fibers.

Further, the polypropylene homopolymer can have a density ranging from about 0.86 grams per cubic centimeter to about 0.94 grams per cubic centimeter, such as from about 0.87 grams per cubic centimeter to about 0.93 grams per cubic centimeter, such as from about 0.88 grams per cubic centimeter to about 0.92 grams per cubic centimeter.

The sheath 101 can further include from about 3 wt. % to about 7 wt. %, such as from about 3.5 wt. % to about 6.5 wt. %, such as from about 4 wt. % to about 6 wt. %, such as from about 4.5 wt. % to about 5.5 wt. % of a charge enhancer package based on the total weight of the sheath component of the multicomponent fiber. In some embodiments, the charge enhancer package can include a combination of a fatty acid metal salt and a fatty acid amide, which are effective in combination to stabilize the electrostatic charge to the nonwoven material, particularly when the nonwoven material is subjected to heat.

In one embodiment, the fatty acid portion of the fatty acid metal salt can be, for example, lauric acid, palmitic acid, stearic acid, oleic acid, etc., and the metal portion of the fatty acid metal salt can be, for example, magnesium, zinc, aluminum, etc. In one particular embodiment, the fatty acid metal salt can be magnesium stearate. Further, the fatty acid metal salt can be present in the sheath in an amount ranging from about 2 wt. % to about 6 wt. %, such as from about 2.5 wt. % to about 5.5 wt. %, such as from about 3 wt. % to about 5 wt. %, such as from about 3.5 wt. % to about 4.5 wt. % based on the total weight of the sheath component of the multicomponent fiber.

Further, the fatty acid amide can be derived from a fatty acid, which can include saturated or unsaturated straight chain carboxylic acids obtained from the hydrolysis of fats. Exemplary fatty acids include lauric acid (dodecanoic acid), myristic acid (tetradecanoic acid), palmitic acid (hexadecanoic acid), stearic acid (octadecanoic acid), oleic acid ((Z)-9-octadecenoic acid), linoleic acid ((Z,Z)-9,12-octadecadienoic acid), linolenic acid ((Z,Z,Z)-9,12,15-octadecatrienoic acid) and eleostearic acid (Z,E,E)-9,11,13-octadecatrienoic acid).

Secondary and tertiary fatty acid amides are also suitable as charge enhancing agents wherein the amide nitrogen is substituted with one or more alkyl groups. Secondary and tertiary fatty acid amides can also be prepared by methods well known in the art, such as by esterification of a fatty acid followed by an amidation reaction with a suitable alkylamine. The alkyl substituents on the amide nitrogen can be straight chain or branched chain alkyl groups and can have between about two and twenty carbon atoms, inclusive, preferably between about two and 14 carbon atoms, inclusive, more preferably between about two and six carbon atoms, inclusive, most preferably about two carbon atoms. In one exemplary embodiment, the fatty acid amide can be a “bis” amide wherein an alkyl chain tethers two nitrogens of two independent amide molecules. For example, alkylene bis-fatty acid amides include alkylene bis(stearamides), alkylene bis(palmitamides), alkylene bis(myristamides) and alkylene bis(lauramides). Typically, the alkyl chain tether includes between about 2 and 8 carbon atoms, inclusive, preferably 2 carbon atoms. The alkyl chain tether can be branched or unbranched. Preferred bis fatty acid amides include ethylene bis(stearamides) and ethylene bis(palmitamides) such as N,N′-ethylenebistearamide and N,N′-ethylenebispalmitamide. Examples of fatty acid amides contemplated for use a part of a charge enhancer package or system with a fatty acid metal salt include stearamide and ethylene bis(stearamide). In certain embodiments of the present disclosure, either N,N′-ethylenebisstearamide or N,N′-ethylenebispalmitamide can be used in the charge enhancer package or system. In other embodiments, the ratio of a C14-C18 fatty acid can be varied from between about 0 to 20% based on the total amount of the bisamides. In still other embodiments, mixtures of N,N′-ethylenebisstearamide and N,N′-ethylenebispalmitamide which fall in the range between about 0 to 100% for each bisamide can be utilized as additive mixtures. In any event, the fatty acid amide can be present in the sheath in an amount ranging from about 0.25 wt. % to about 2 wt. %, such as from about 0.5 wt. % to about 1.75 wt. %, such as from about 0.75 wt. % to about 1.5 wt. %, such as from about 1 wt. % to about 1.25 wt. % based on the total weight of the sheath component of the multicomponent fiber.

Further, the ratio of the fatty acid metal salt to the fatty acid amide can range from about 3:1 to about 5:1, such as from about 3.25:1 to about 4.75:1, such as from about 3.5:1 to about 4.5:1, such as from about 3.75:1 to about 4.25:1 in some embodiments. Without intending to be limited by any particular theory, the present inventors have found that this ratio supports improvements in BFE, PFE, and pressure drop or differential post-sterilization with gamma irradiation when utilized in the bicomponent fibers and nonwoven materials of the present disclosure.

Meanwhile, the sheath 101 can also include from about 2 wt. % to about 6 wt. %, such as from about 2.5 wt. % to about 5.5 wt. %, such as from about 3 wt. % to about 5 wt. %, such as from about 3.5 wt. % to about 4.5 wt. % of an antioxidant based on the total weight of the sheath component of the multicomponent fiber. In some embodiments, the antioxidant can include one or more of a partially-hindered phenolic antioxidant, such as benzenepropanoic acid, 3-(1,1-dimethylethyl)-4-hydroxy-5-methyl-, 1,1′-[2,4,8,10-tetraoxaspiro[5.5]undecane-3,9-diylbis(2,2-dimethyl-2, 1-ethanediyl)] ester (CAS 90498-90-1), a hindered amine antioxidant, such as dimethyl succinate polymer with 4-hydroxy-2,2,6,6,-tetramethyl-1-piperidineethanol (CAS 65447-77-0) or poly(6-((1,1,3,3-tetramethylbutyl)amino)-s-triazine-2,4diyl) ((2,2,6,6-tetramethyl-4-piperidinyl)imino)) hexamethylene ((2,2,6,6-tetramethyl-4-piperidinyl)imino) (CAS 70624-18-9), and/or a phosphite-based antioxidant containing spiro rings, such as 2,4,8,10-Tetraoxa-3,9-diphosphaspiro[5.5]undecane, 3,9-bis[2,6-bis(1,1-dimethylethyl)-4-methylphenoxy]-(CAS 80693-00-1). One commercially available antioxidant is 21SAM0938, available from Standridge Color Corporation.

### Core

In some embodiments, the fibers 100 from which the nonwoven material is formed can have a sheath-core arrangement where the core 102 can include from about 80 wt. % to about 98 wt. %, such as from about 84 wt. % to about 96 wt. %, such as from about 88 wt. % to about 92 wt. % of a thermoplastic polyester (e.g., polyethylene terephthalate) based on the total weight of the core component of the multicomponent fiber. In one embodiment, the thermoplastic polyester can include a polyethylene terephthalate homopolymer, a polyethylene terephthalate copolyester, or a combination thereof, where polyethylene terephthalate (PET) is a semi-crystalline polymer produced through the polymerization of ethylene glycol and terephthalic acid. In some embodiments, the thermoplastic polyester can have a low intrinsic viscosity ranging from about 0.50 dL/g to about 0.65 dL/g, such as from about 0.52 dL/g to about 0.62 dL/g, such as from about 0.54 dL/g to about 0.60 dL/g. Without intending to be limited by any particular theory, the present inventors have found that the use of a low intrinsic viscosity polyester decreases the number of entanglements of the polymer chains, resulting in a less stiff material compared to higher intrinsic viscosity polymers, where the less stiff material is more amenable to the formation of fibers. Further, lower intrinsic viscosity can lead to smaller fiber sizes, which can increase surface area and hence filtration capabilities of the resulting material. Moreover, the present inventors have found that because PET and polypropylene have such differing properties, controlling the intrinsic viscosity of the PET as outlined in the ranges above, while also using a lower melt flow rate polypropylene (discussed in more detail above) in specific combination with the PET, allows for improved compatibility between the two polymers in order to optimize flow and mixing characteristics of the materials that were extruded through a die together to form the biocomponent fibers.

The core 102 can further include from about 2 wt. % to about 6 wt. %, such as from about 2.5 wt. % to about 5.5 wt. %, such as from about 3 wt. % to about 5 wt. %, such as from about 3.5 wt. % to about 4.5 wt. % of a charge enhancer package based on the total weight of the core component of the multicomponent fiber.

In one embodiment of the present disclosure, the charge enhancer package can include a fatty acid metal salt. In one embodiment, the fatty acid portion of the fatty acid metal salt can be, for example, lauric acid, palmitic acid, stearic acid, oleic acid, etc., and the metal portion of the fatty acid metal salt can be, for example, magnesium, zinc, aluminum, etc. In one particular embodiment, the fatty acid metal salt can be magnesium stearate. In another embodiment, the charge enhancer package can include barium titanium dioxide. Further, it should be understood that the charge enhancer package can include both a fatty acid metal salt and barium titanium dioxide in combination. The fatty acid metal salt and the barium titanium dioxide can be effective in combination to stabilize the electrostatic charge to the nonwoven material, particularly when the nonwoven material is subjected to heat. When used in combination, the weight ratio of the fatty acid metal salt to the barium titanium dioxide can range from about 0.2:1 to about 6:1, such as from about 0.3:1 to about 5:1, such as from about 0.4:1 to about 4:1, such as about 0.5:1 to about 3:1 and vice versa.

Commercially available charge enhancers include from PTM140357 and PTM140359, available from Techmer, charge enhancers 66248-D25-200, 66249-D25-200, and 66250-D25-200 available from Americhem, and charge enhancers available from Sukano Polymers Corporation.

Meanwhile, the core 102 can also include from about 3 wt. % to about 9 wt. %, such as from about 3.5 wt. % to about 8.5 wt. %, such as from about 4 wt. % to about 8 wt. %, such as from about 4.5 wt. % to about 7.5 wt. % of an antioxidant package based on the total weight of the core component of the multicomponent fiber.

In one embodiment of the present disclosure, the antioxidant package can include can include one or more of a phenolic antioxidant, such as 2,2′-methylenebis-(4-methyl-6-tertiary-butylphenol) (CAS 119-47-1), a hindered amine antioxidant, such as dimethyl succinate polymer with 4-hydroxy-2,2,6,6,-tetramethyl-1-piperidineethanol (CAS 65447-77-0) or poly(6-((1,1,3,3-tetramethylbutyl)amino)-s-triazine-2,4diyl) ((2,2,6,6-tetramethyl-4-piperidinyl)imino)) hexamethylene ((2,2,6,6-tetramethyl-4-piperidinyl)imino) (CAS 70624-18-9), a phenyl phosphite antioxidant, and/or a hydroxyphenyl-triazine antioxidant. One commercially available antioxidant is AO 2246 or AO 1. Other commerically available antioxidants include PTM140354 and PTM140389, available from Techmer, and antioxidants available from Americhem and Sukano Polymers Corporation.

Referring to FIG. 2, a nonwoven material 200 is illustrated that can be formed from a plurality of multicomponent fibers 100 (e.g., bicomponent fibers) formed as described above. In some embodiments, the nonwoven material 200 can be a meltblown material, although it is to be understood that the material can be any suitable type of nonwoven material, such as a spunbond or a meltblown-spunbond-meltblown (SMS) laminate. Prior to forming it into a product, the nonwoven material can then be subjected to an electret treatment before being formed into a desired product that is then packaged for sterilization via gamma irradiation. It has been found that electret treatment increases the BFE of a fabric. Electret treatment is described, for example, in U.S. Pat. No. 5,592,357 to Rader, et al. Electret treatment is used to produce an intense corona current at reduced voltages to help reduce the potential for arcing and provide a more efficient, stable discharge at atmospheric pressure, for electrostatically charging an advancing web or film. Once ionization occurs, excess charged particles cannot be lost until they collide with a solid body, preferably the remaining electrode, achieving the desired result. It has been found that this applies to both AC and DC voltages.

Placement of a thin, non-electron absorbing gas layer in the vicinity of an electrode is advantageously accomplished by various means. For example, the charging bar can be replaced with a longitudinally extending tube having spaced apertures for delivering a gas to the discharge-forming elements of the electrode. These discharge-forming elements can include either a series of pins that extend through the spaced apertures of the tube, or a series of nozzles that project from the surface of the tube. In either case, this places the gas in the vicinity of the pins, or the nozzles, which in turn receive appropriate biasing voltages for developing the electric field that is to produce the improved discharge. Alternatively, the charging shell can be replaced with a hollow body that similarly incorporates a series of apertures, and a cooperating series of pins or nozzles, to achieve a similar result.

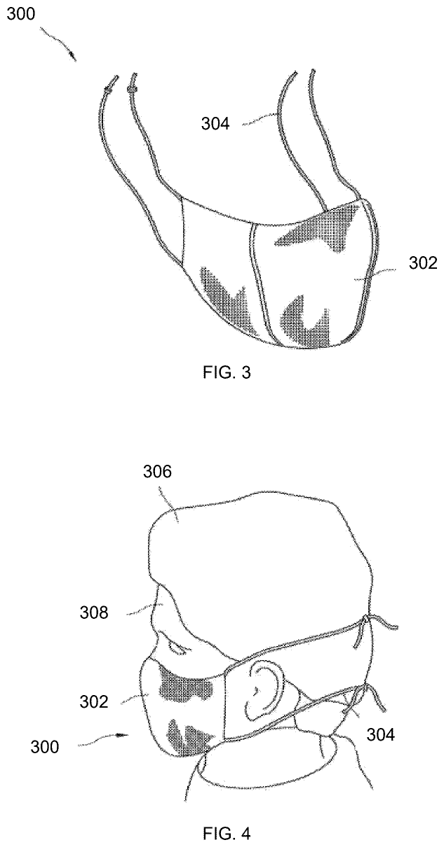

After electret treatment, the nonwoven material can be formed into various products. FIGS. 3 and 4 show a face mask 300 which may be formed from nonwoven material of the present disclosure. The face mask 300 includes a body portion 302 that is configured to be placed over the mouth and at least part of the nose of the user 308 such that the air exchanged through normal respiration passes through the body portion 302 of the face mask 300. It is to be understood, however, that the body portion 302 can be of a variety of styles and geometries, such as, but not limited to, flat half mask, pleated face masks, cone masks, flat folded personal respiratory devices, duckbill style mask, trapezoidally shaped masks, etc. The body portion 302 may be configured to have one or more horizontal pleats, one or more vertical pleats, or no pleats, where such designs are generally known in the art. The face mask 300 can therefore isolate the mouth and the nose of the user 308 from the environment. The face mask 300 can be attached to the user 308 by a pair of tie straps 304 which are wrapped around the head of the user 308 (and a hair cap 306 if worn by the user 308) and are connected to one another. It is to be understood, however, that other types of fastening arrangements may be employed in accordance with various exemplary embodiments of the present disclosure. For instance, instead of the tie straps 304, the face mask 300 may be attached to the user 308 by ear loops, elastic bands wrapping around the head, or a hook and loop type fastener arrangement, or the face mask 300 may be wrapped as a single piece around the head of the user 308 by an elastic band. The face mask 300 may also be directly attached to the hair cap 306.

Additionally, the configuration of the face mask 300 may be different in accordance with various exemplary embodiments. In this regard, the face mask 300 may be made such that it covers both the eyes, hair, nose, throat, and mouth of the user. As such, the present disclosure is not limited to only face masks 300 that cover only the nose and mouth of the user 308.

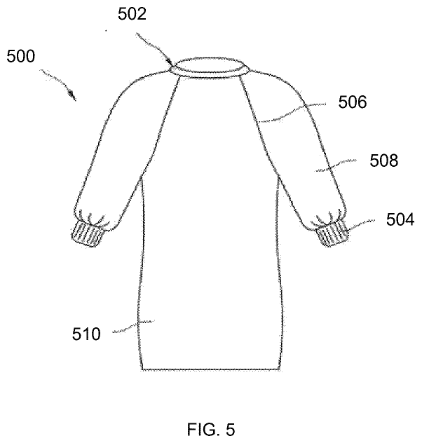

Meanwhile, FIG. 5 shows a front view of a surgical gown 500 that can be formed from the nonwoven material contemplated by the present disclosure. The gown 500 can include a collar 502, cuffs 504, and shoulder seams 506 that link the sleeves 508 to the main body 510. Other exemplary articles that can be formed from the nonwoven material of the present disclosure include surgical drapes or any other PPE. Further, the multiple layers of nonwoven material can be used to form the aforementioned face masks, surgical gowns, drapes, bouffant caps, etc. and may be joined by various methods, including adhesive bonding, thermal point bonding, or ultrasonic bonding.

The present disclosure is further described in the following Example, which does not limit the scope of the disclosure described in the claims.

### Example 1

### Test Methods

### 1. Particle Filtration Efficiency

In Particle Filtration Efficiency testing according to ASTM F2299, the filtration efficiency of filter media materials against sub-micron particles cannot be determined using viable challenge particles. This procedure involves the generation of a particle aerosol using NIST traceable polystyrene microspheres with a Particle Measurement Systems (PMS) Model PG-100 particle generator. The particles are counted with a PMS Model LASAIR II-110 or 310 laser particle counter.

The latex particles used in this procedure have a narrow standard deviation and the design of the PMS particle generator produces a consistent particle challenge. Testing is conducted at a single particle size. ASTM F2100 specifies a challenge particle size of 0.1 μm. The PMS particle counter is an optical laser based device and operates at a flow rate of 1 cubic foot per minute (CFM) or 28.3 liters per minute (LPM).

### 2. Bacterial Filtration Efficiency

The Bacterial Filtration Efficiency test according to ASTM F2101 determines the filtration efficiency by comparing the bacterial control counts to test article effluent counts. The test is conducted using Staphylococcus aureus as the challenge organism. After the filtration media is preconditioned, a liquid suspension of S. aureus is aerosolized and delivered to the filtration media at a constant flow rate of 28.3 liters per minute (LPM) or 1 cubic foot per minute (CFM).

The aerosol droplets are drawn through a six-stage Andersen sampler for collection. The number of bacterial aerosol droplets contacting the filter media is determined by conducting challenge controls without filter medium in the test system. Challenge controls are maintained at 1700-3000 colony-forming units (CFU) with a mean particle size (MPS) of 3.0±0.3 μm. This allows filtration efficiencies to be reported up to >99.9%.

### 3. Differential Pressure (dP)

There are a number of methods of characterizing the air filtration efficiencies of nonwoven webs. For this example, the typical air flow rate was 8 liters per minute, with a relative humidity of 85% plus or minus 5%, at a temperature of 25° C. plus or minus 5° C. The test method complies with EN 14683:2019, Annex C. The performance or efficiency of a filter medium is expressed as the percentage of sodium chloride particles that penetrate the filter.

In Example 1, the various samples shown in Table 1 below were tested for PFE, BFE, and dP according to the methods described above. The results are shown in Table 2, Table 3, and Table 4, respectively.

TABLE 1
Formulations

| | Basis | | | Sheath Additive Package | | | | Core Additive Package | |
|---|---|---|---|---|---|---|---|---|---|
| | Weight | Sheath | Sheath | Charge | | Core | Core | Charge | |
| Sample | (gsm) | Polymer | % | Enhancer | Antioxidant | Polymer | % | Enhancer | Antioxidant |
| 1 | 20.3 | Polypropylene | 100 | Magnesium Stearate 2 wt. % | N/A | N/A | 0 | N/A | N/A |
| (Control) | | 1550 g/10 min | | Ethylene bis(stearamide) | | | | | |
| | | MFR | | 1 wt. % | | | | | |
| 2 | 23.9 | Polypropylene | 50 | Magnesium Stearate 4 wt. % | 21SAM0938 | Polyethylene | 50 | PTM140357 | PTM140354 |
| | | 1550 g/10 min | | Ethylene bis(stearamide) | 4 wt. % | terephthalate | | 4 wt. % | 5 wt. % |
| | | MFR | | 1 wt. % | | crystallized | | | |
| | | | | | | homopolymer | | | |
| | | | | | | 0.59 dl/g IV | | | |
| 3 | 24.3 | Polypropylene | 50 | Magnesium Stearate 4 wt. % | 21SAM0938 | Polyethylene | 50 | PTM140357 | PTM140388 |
| | | 1550 g/10 min | | Ethylene bis(stearamide) | 4 wt. % | terephthalate | | 4 wt. % | 7 wt. % |
| | | MFR | | 1 wt. % | | crystallized | | | |
| | | | | | | homopolymer | | | |
| | | | | | | 0.59 dl/g IV | | | |
| 4 | 24.8 | Polypropylene | 50 | Magnesium Stearate 4 wt. % | 21SAM0938 | Polyethylene | 50 | PTM140359 | PTM140389 |
| | | 1550 g/10 min | | Ethylene bis(stearamide) | 4 wt. % | terephthalate | | 4 wt. % | 7 wt. % |
| | | MFR | | 1 wt. % | | copolyester | | | |
| | | | | | | 0.56 dl/g IV | | | |

TABLE 2
PFE Testing
Pre-Sterilization and Post-Sterilization Particle
Filtration Efficiency Testing Results

| | Particle | Particle | |
|---|---|---|---|
| | Filtration | Filtration | Δ Particle |
| | Efficiency | Efficiency | Filtration |
| | (PFE) % | (PFE) % | Efficiency |
| Sample | Pre-Sterilization | Post-Sterilization | (PFE) % |
| 1 - Control | 99.81 | 88.00 | −13.42 |
| 2 | 99.67 | 96.89 | −2.87 |
| 3 | 99.63 | 96.30 | −3.46 |
| 4 | 99.68 | 95.82 | −4.03 |

TABLE 3
BFE Testing
Pre-Sterilization and Post-Sterilization Bacterial
Filtration Efficiency Testing Results

| | Bacterial | Bacterial | |
|---|---|---|---|
| | Filtration | Filtration | Δ Bacterial |
| | Efficiency | Efficiency | Filtration |
| | (BFE) % | (BFE) % | Efficiency |
| Sample | Pre-Sterilization | Post-Sterilization | (BFE) % |
| 1 - Control | 99.80 | 91.30 | −9.31 |
| 2 | 99.53 | 95.26 | −4.48 |
| 3 | 99.29 | 95.46 | −4.01 |
| 4 | 99.33 | 96.69 | −2.73 |

TABLE 4
dP Testing
Pre-Sterilization and Post-Sterilization Differential
Pressure Testing Results

| | dP | dP | Δ dP |
|---|---|---|---|
| | (mm H₂O/cm²) | (mm H₂O/cm²) | (mm H₂O/cm²) |
| Sample | Pre-Sterilization | Post-Sterilization | % |
| 1 - Control | 2.76 | 2.69 | −2.60 |
| 2 | 3.88 | 3.53 | −9.92 |
| 3 | 3.77 | 3.64 | −3.57 |
| 4 | 4.78 | 4.01 | −19.20 |

As shown from Table 2 above, compared to a control sample meltblown material formed from a plurality of polypropylene monofilaments, the meltblown materials formed from a plurality of bicomponent fibers having a polypropylene sheath and a polyethylene terephthalate core exhibited improved levels of particle filtration efficiency (PFE) % post-sterilization. While the control sample showed a more than 13% decrease in PFE %, samples 2-4, which contained a polypropylene sheath and a polyethylene terephthalate core, showed only minimal reductions in PFE %. Moreover, as shown in Table 3, samples 2-4 also exhibited lower % changes (decreases) in bacterial filtration efficiency (BFE) % post-sterilization compared to the control sample, indicating improved performance compared to the control. Lastly, referring to Table 4, overall, samples 2-4 showed a decrease in their dP post sterilization, indicating improved breathability compared to before sterilization. In summary, the data shows the specific combination of a polypropylene sheath and a polyethylene terephthalate core to form meltblown bicomponent fibers finds a balance between comfort/breathability and filtration efficiency that is not seen with nonwoven materials formed from polypropylene monofilament fibers.

While various embodiments of the present disclosure have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the present disclosure, but instead as merely intended to highlight alternative or additional features that may or may not be utilized in a particular embodiment of the present disclosure. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments. For instance, when a plurality of ranges are provided, any combination of a minimum value and a maximum value described in the plurality of ranges are contemplated by the present disclosure. For example, if ranges of ‘from about 20% to about 80%’ and ‘from about 30% to about 70%’ are described, a range of ‘from about 20% to about 70%’ or a range of ‘from about 30% to about 80%’ are also contemplated by the present disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and ‘one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more” or ‘at least one’ and indefinite articles such as ‘a’ or ‘an’ (e.g., ‘a’ and/or ‘an’ should typically be interpreted to mean ‘at least one’ or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the terms ‘about,’ ‘approximately,’ or ‘generally.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches. As used herein, the terms ‘about,’ ‘approximately,’ or ‘generally,’ when used to modify a value, indicate that the value can be raised or lowered by 5% and remain within the disclosed embodiment.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

What is claimed is:

1. An article comprising:
a nonwoven material, the nonwoven material comprising a plurality of bicomponent fibers each having a core and a sheath,
wherein the core comprises from about 80 wt. % to about 98 wt. % of an olefin homopolymer, from about 3 wt. % to about 7 wt. % of a first charge enhancer package, and from about 2 wt. % to about 6 wt. % of a first antioxidant package, based on the total weight of the core,
wherein the sheath comprises from about 80 wt. % to about 98 wt. % of a thermoplastic polyester, from about 2 wt. % to about 6 wt. % a second charge enhancer package, and from about 3 wt. % to about 9 wt. % of a second antioxidant package based on a total weight of the sheath, and
wherein the nonwoven material exhibits a reduction in particle filtration efficiency after sterilization by gamma irradiation at a level of about 20 kilograys to about 30 kilograys of less than about 5%.

2. The article of claim 1, wherein the olefin homopolymer comprises polypropylene.

3. The article of claim 1, wherein the olefin homopolymer has a melt flow rate ranging from about 400 grams/10 minutes to about 1700 grams/10 minutes as determined at 2.16 kg and 230° C.

4. The article of claim 1, wherein the olefin homopolymer has a density ranging from about 0.86 grams/cm³ to about 0.94 g/cm³.

5. The article of claim 1, wherein the thermoplastic polyester comprises polyethylene terephthalate.

6. The article of claim 1, wherein the thermoplastic polyester has an intrinsic viscosity ranging from about 0.50 dL/g to about 0.65 dL/g.

7. The article of claim 1, wherein the first charge enhancer package comprises a fatty acid metal salt and a fatty acid amide.

8. The article of claim 7, wherein the fatty acid metal salt comprises a fatty acid portion and a metal portion, wherein the fatty acid portion comprises lauric acid, palmitic acid, stearic acid, oleic acid, or a combination thereof and the metal portion comprises magnesium, zinc, aluminum, or a combination thereof.

9. The article of claim 7, wherein a ratio of the fatty acid metal salt to the fatty acid amide ranges from about 3:1 to about 5:1.

10. The article of claim 1, wherein the first antioxidant package comprises a partially-hindered phenolic antioxidant, a hindered amine antioxidant, a phosphite-based antioxidant, or a combination thereof.

11. The article of claim 1, wherein the second charge enhancer package comprises a fatty acid metal salt, barium titanium dioxide, or a combination thereof.

12. The article of claim 1, wherein the second antioxidant package comprises a phenolic antioxidant, a hindered amine antioxidant, a phenyl phosphite antioxidant, a hydroxyphenyl-triazine antioxidant, or a combination thereof.

13. The article of claim 1, wherein the nonwoven material exhibits a particle filtration efficiency after sterilization by gamma irradiation at a level of about 20 kilograys to about 30 kilograys of at least about 95%.

14. The article of claim 1, wherein the nonwoven material exhibits a bacterial filtration efficiency after sterilization by gamma irradiation at a level of about 20 kilograys to about 30 kilograys of at least about 94%.

15. The article of claim 1, wherein the nonwoven material exhibits a differential pressure (dP) after sterilization by gamma irradiation at a level of about 20 kilograys to about 30 kilograys of less than about 5 mm water/cm².

16. The article of claim 1, wherein the nonwoven material comprises a meltblown material.

17. The article of claim 16, wherein the nonwoven material comprises a laminate, wherein the laminate comprises the meltblown material.

18. The article of claim 17, wherein the laminate further comprises a spunbond material.

19. The article of claim 1, wherein the nonwoven material is electret-treated prior to sterilization.

20. The article of claim 1, wherein the article comprises a face mask, a surgical cap, or a surgical gown.
